(19) United States
(12) Patent Application Publication
(10) Pub. No.: US 2024/0416055 A1
(43) Pub. Date: Dec. 19, 2024

(54) AGENT DELIVERY DEVICE, AGENT DELIVERY SYSTEM, AND METHOD OF DELIVERING AN AGENT

(71) Applicant: ASAP BreatheAssist Pty Ltd, Cremorne (AU)

(72) Inventors: Michael Ralph Burgess Johnson, Hawthorn (AU); Justin Robert Armistead, The Basin (AU); Toby James Hartley, Ferntree Gulley (AU)

(21) Appl. No.: 18/015,951

(22) PCT Filed: Jul. 13, 2021
(86) PCT No.: PCT/AU2021/050746

§ 371 Date: Jan. 12, 2023

(30) Foreign Application Priority Data
Jul. 13, 2020 (AU) 2020902417
Sep. 29, 2020 (AU) 2020903519
Oct. 16, 2020 (AU) 2020903763
Nov. 19, 2020 (AU) 2020904284

(51) Int. Cl. A61M 15/08; A61M 21/00; A61M 21/02; A61M 31/00; A61M 37/00

(52) U.S. Cl. CPC A61M 15/085; A61M 21/02; A61M 31/00; A61M 37/0015; A61M 2021/0077; A61M 2037/0023; A61M 2210/0618

(57) ABSTRACT

The present disclosure provides an agent delivery device (1) for delivery of a therapeutic agent or medicinal agent to an individual. The agent delivery device (1) comprises: a frame (2) having at least one frame member (4) configured to be received and retained or accommodated within a nostril of the individual; and a delivery element (8) provided on and/or carried by the at least one frame member (4) for delivering the agent, the agent preferably being either in a liquid or semi-solid form or contained in a liquid or gel medium. The delivery element (8) is provided on and/or carried by the frame member such that, when the frame member is accommodated in a nostril, the delivery element (8) contacts or engages with an inner surface (e.g., tissue) of the nostril to deliver the agent to the individual, e.g., percutaneously. In this way, the agent delivery device (1) has a delivery element (8) provided on and/or carried by a frame member (4) received and retained or accommodated within a nostril of the individual to deliver the agent in situ there. The disclosure also provides an agent delivery kit, an agent delivery system, and associated methods.

### CROSS-REFERENCE TO RELATED APPLICATIONS

This application is a national stage filing under 35 U.S.C. § 371 of International Application No. PCT/AU2021/050746, filed Jul. 13, 2021, which claims priority to Australian Application No. 2020904284, filed Nov. 19, 2020, Australian Application No. 2020903763, filed Oct. 16, 2020, Australian Application No. 2020903519, filed Sep. 29, 2020, and Australian Application No. 2020902417, filed Jul. 13, 2020. The entire contents of these applications are incorporated herein by reference in their entirety.

### TECHNICAL FIELD

The disclosure relates to an agent delivery device, and especially to a device for delivering an agent nasally to a user, as well as to an agent delivery system and a method of delivering an agent.

The present disclosure has particular application to delivery of a medicinal agent or a medicament to the nostril(s) of an individual, and it will be convenient to describe the subject of this disclosure in this exemplary context. It will be appreciated, however, that the disclosure is not limited to this particular application but may be used to deliver an agent for a range of different purposes.

### SUMMARY OF THE DISCLOSURE

According to one aspect, the present disclosure provides an agent delivery device for delivery of a therapeutic or medicinal agent to an individual, the agent delivery device comprises: a substrate configured to be received and retained or accommodated within a nostril of the individual; and delivery means (e.g. a delivery element) provided on or carried by the substrate for delivering the agent, with the agent preferably either in a liquid or gel form or contained in a liquid or gel medium, although optionally able to be provided or contained in a film-like form or in a powder. The delivery means or delivery element is provided on or carried by the substrate such that, when the substrate is retained or accommodated within a nostril, the delivery means/delivery element contacts or engages an inner wall or surface (e.g., the tissue) of the nostril to deliver the agent to the individual, preferably percutaneously.

Thus, the disclosure provides an agent delivery device with a delivery means or a delivery element, which may be in the form of a pad or swab, provided on and/or carried by a frame member that is received and retained or accommodated within a nostril of the individual to deliver the agent there. Thus, the pad or swab can be inserted or introduced into the nostril by an individual him- or herself. It does not require assistance by another person or a skilled medical practitioner and is highly suited to use outside of a clinical practice or a clinical environment, such as, for example, at home.

It will be noted that the reference to an “agent” is understood to include any active ingredient with medicinal or therapeutic properties that may be comprised of or contained or carried in a liquid, gel, semi-solid, solid, or powder medium that may be delivered to an individual, particularly to the nose (e.g., the nasal cavity) of the individual. Thus, the agent delivered to an individual will be most typically in a liquid or gel form or contained or carried in a liquid or gel medium for delivery to the nose.

In a preferred embodiment, the substrate forms a frame of the agent delivery device for supporting the delivery means or delivery element provided or carried thereon. Preferably, the frame comprises at least one frame member configured to be received and retained or accommodated within the nostril of the individual, and the delivery means or delivery element is provided on and/or carried by the frame member. As noted above, the substrate is configured to be received and retained within an anterior nasal cavity, e.g., within the nares or the nasal vestibule, for delivering the agent. In this way, the agent delivery device is preferably configured to be worn by the individual.

In at least one embodiment, therefore, the present disclosure provides an agent delivery device for delivery of a therapeutic agent or medicinal agent to an individual. The agent delivery device comprises: a frame having at least one frame member configured to be received and retained or accommodated within a nostril of the individual; and a delivery element provided on and/or carried by the at least one frame member for delivering the agent, the agent preferably being either in a liquid or semi-solid form or contained in a liquid or gel medium. The delivery element is provided on and/or carried by the frame member such that, when the frame member is accommodated in a nostril, the delivery element contacts or engages with an inner surface (e.g. tissue) of the nostril to deliver the agent to the individual; e.g. percutaneously. In this way, the agent delivery device has a delivery element provided on and/or carried by a frame member that is received and retained or accommodated within a nostril of the individual to deliver the agent in situ there.

In a preferred embodiment, the at least one frame member on which the delivery means or delivery element is provided or carried is configured to be received and retained or accommodated within the anterior nasal cavity, e.g. within the nares or nasal vestibule, for delivering the agent to the nasal tissue. In this regard, the scientific literature indicates that the nares are valid for the delivery of an agent or medicament via a nasal swab. This region of the nose is also directly accessible by the individual him-/herself and simplifies the introduction and positioning of the agent delivery device within the nostril.

In a preferred embodiment, the delivery means/delivery element is impregnated with the therapeutic agent or medicinal agent such that, when the substrate or the frame member(s) of the device is/are retained or accommodated in the nostril(s) and the delivery means/delivery element(s) contacts or engages with an inner wall or surface (i.e. tissue) of the nostril(s), the therapeutic or medicinal agent impregnated on the delivery means or delivery element is delivered to the individual; for example, by percutaneous absorption.

In a preferred embodiment, each delivery element comprises a delivery material for holding and delivering the agent. As noted above, each delivery element is preferably in the form of a pad or swab. Thus, the delivery material may be comprised of fibres, such as flocked fibres, compressed fibres, fibre sheet, knitted fibres, and/or of a foam. In the case of the delivery material being comprised of fibres, the delivery material may, e.g. be selected from a group consisting of cotton, rayon, calcium alginate, polypropylene and polyethylene (e.g. polyester). In the case of the delivery material being comprised of a foam, the material may, for example, comprise a urethane foam. In the context of this disclosure, the “delivery” of the agent involves bringing or conveying the agent for uptake of the agent by the individual. In this regard, the agent held by the delivery element may be conveyed to the individual by, for example, capillary action in the delivery material as the agent is gradually absorbed percutaneously.

As noted above, in a preferred embodiment each delivery element may be in the form of a pad of delivery material, such as a pad of compressed fibres, an extruded pad of fibres, a pad of knitted fibres, or a foam pad. In an alternative embodiment, the delivery element may be in the form of a sleeve of delivery material, e.g. a sleeve of knitted fibres, a flocked sleeve, an extruded sleeve, or a foam sleeve. As also noted above, the delivery element may optionally be integrated or integrally formed with the frame member upon which it is provided or carried. Where, for example, the delivery element comprises a foam material, it could be an outer region or layer of a frame member comprised of the same foam material. In addition, or alternatively, the or each delivery element preferably comprises one or more needles (e.g. micro-needles) projecting from a surface thereof for contact and/or engagement with the inner surface or tissue of the nasal cavity to promote delivery of the agent; for example, percutaneously.

In a preferred embodiment, the at least one frame member on which the delivery means, e.g., the delivery element, is provided and/or carried is preferably elongate and preferably extends as a rib which may follow a profile or curvature of the inner surface of the nostril. In this regard, the frame member or rib member may be formed to complement a surface profile or curvature of the nasal cavity, which naturally enhances comfort for the wearer and also serves to improve contact between the delivery element and the inner surface or tissue of the nasal cavity. To this end, the frame member or rib member of the agent delivery device on which the delivery element is provided or carried preferably has a curved or looped configuration. In this regard, the elongate frame member (or rib member) may exhibit an arched profile which approximates at least a portion of a circle, an ellipse or a parabola. This enables the frame member to be received and to seat within the nostril comfortably and consistently, which is important for achieving both consistency in use and consistency in agent delivery. The delivery element is typically provided and/or carried on a surface or region of the frame member that faces the tissue of the nasal cavity. In the context of wearer comfort and ease of introduction into the nostril, the elongate frame member is preferably relatively soft, resiliently flexible and is preferably biased into contact with the nostril so that the delivery element engages with tissue of the nasal cavity when accommodated therein.

In a preferred embodiment, the at least one frame member on which the delivery element is provided or carried is configured to be broader or wider in a region thereof to be located deeper within the nasal cavity or nares. In this way, the frame member provides a larger surface area for bringing the delivery element provided or carried thereon into contact with nasal tissue in an area of the nasal cavity or nares likely to provide a greater agent load to be delivered. For similar reasons, the at least one frame member may be divided, splayed or open in this region to provide a greater surface area for supporting the delivery means (e.g., comprising fibres, especially flocked fibres), thereby to increase a potential agent load.

In a preferred embodiment, the frame comprises a pair of frame members, each of which is configured to be accommodated within a respective one of the nostrils of the individual. Thus, the device preferably comprises a respective delivery means/delivery element provided on and/or carried by each of those frame members such that each delivery means contacts, bears against or engages with the inner wall or tissue of a respective one of the nostrils to deliver the agent. As will be appreciated, the delivery device is thus preferably configured with frame members and delivery elements designed to be received and retained in both nostrils of the individual simultaneously. In view of greater contact area provided by both nasal cavities, it is beneficial if the agent is able to be delivered to both nasal cavities simultaneously. It is preferable that, when deployed or accommodated within the nostrils, the agent delivery device will not fully obscure or block the nasal passages but will still allow the individual to breathe through the nose. Nevertheless, it may possibly be more comfortable for the individual to breathe through the mouth during use of the agent delivery device.

In a preferred embodiment, the agent delivery device is wearable; that is, the device is configured to be worn by the individual such that the delivery element, which is provided on and/or carried by the at least one frame member, is received and retained or accommodated within the nostril of the individual for an extended and/or predetermined period of time for agent delivery. In this regard, the agent delivery time is preferably in the range of about 5 minutes to about 8 hours, and more preferably in the range of about 10 minutes to about 4 hours. Depending on the condition of the individual to whom the agent is being delivered and the nature of the agent, however, the delivery time could be lower, but will typically be at least about 2 or 3 seconds, preferably at least about 10-30 seconds, and more typically at least about one or two minutes. The longer agent delivery time which arises or is achieved by ‘wearability’ of the device can increase the ‘agent load’ being delivered by the element of the device.

In a preferred embodiment, the agent delivery device includes a timing indicator or means for indicating to the individual or wearer when a predetermined agent delivery time has elapsed. In this regard, the indicator may be provided on a part of the frame that is external of the nostrils, and therefore visible, during use of the device. In a particular preferred embodiment, the timing indicator comprises a patch that is configured to change appearance (e.g., to change colour) over time. For example, the indicator patch could include a substance selected to react with air and/or light over the predetermined time period in order to change colour over that time. Upon commencing use of the device, therefore, the indicator patch may be initially exposed to air and/or light (e.g., by removing a protective covering, such as an adhesive cover or label). After the predetermined agent delivery time has elapsed, the indicator patch will have changed colour due to exposure to air and/or light to indicate to the user or wearer that the agent delivery device can now be removed. In this way, the device can be designed to achieve consistency of use by individuals.

In a preferred embodiment, the agent delivery device comprises a supply of the agent supported on the substrate or frame of the device. In this regard, the supply may be arranged in communication with the delivery means/delivery element for providing or conveying the agent thereto. In this way, as the medicinal or therapeutic agent is delivered to and taken up by the individual at a delivery means/delivery element, additional agent can then be conveyed from the supply to the delivery means/delivery element, thereby to replenish or recharge that delivery means/delivery element. The supply of the agent may be configured as or comprise a reservoir for holding an amount of the agent, preferably in a liquid or gel form. In such a case, the reservoir may be connected in fluid communication with the delivery means or delivery element for conveying the agent thereto from the reservoir. The supply is desirably configured to hold a predetermined amount of the agent (e.g., for a particular dosage during the predetermined time period). Furthermore, the supply may preferably be configured to be removably mounted or replaceable on the frame of the device for recharging the supply of the agent. In this context, the supply reservoir may be in the form of a receptacle filled with the agent, which receptacle may be removable or replaceable on the frame of the device. The agent delivery device may be configured to convey the agent from the supply to the delivery element passively (e.g., via capillary action) or actively (e.g. via pump means, such as a micro-pump). The active transfer or conveyance of the agent may be activated by a user/wearer of the device. For example, the device may incorporate a pump means for activation or manual operation by the user/wearer. In this regard, the receptacle filled with the agent may be deformable upon application of manually pressure to forcibly transfer the agent from the reservoir to the delivery elements.

In an alternative embodiment, each delivery element of the agent delivery device may be designed or adapted to be separated or removed from the frame to re-supply the agent thereon. For example, in one particular embodiment, the or each delivery element may be configured in the form of a sleeve of delivery material configured to extend over an outer periphery of the respective frame member on which it is provided and/or carried. The sleeve of delivery material may thus be removable from the frame member (e.g. may be pulled off or drawn off the frame member) for re-supplying with the agent thereon.

In another embodiment, the or each delivery element of the agent delivery device may be integrated or integrally formed with the frame member upon which it is provided or carried. In this regard, the delivery element may be comprised of the same material as the frame member and may, for example, form an outer region or layer of the frame member for receiving and applying the agent. Thus, in certain embodiments, the material of the frame member may potentially perform a dual role as a delivery element also.

In a preferred embodiment, the frame of the agent delivery device includes a handle portion for manual handling of the device by the individual. In this regard, the or each frame member on which the delivery element(s) is/are provided or carried desirably extend/s from the handle portion. The handle portion thus enables a user to manipulate the agent delivery device by hand without handling and potentially contaminating the delivery elements; e.g. before delivery of the agent. The handle portion is preferably arranged centrally of the device and may be located between the two frame members configured to be received and accommodated within the respective nostrils. In order to provide greater clarity for the individual user which part(s) of the agent delivery device should and should not be touched by hand, the device may be colour coded. That is, the handle portion may be clearly marked or coloured to identify that manual handling is permitted, and other parts of the device may be differently marked or coloured to reflect that manual handling of those parts is to be avoided. The timing indicator described above for providing an indication of the agent delivery time may preferably be provided on the handle portion. The handle portion is optionally removably connected to the frame or to the or each frame member on which a respective delivery means is provided or carried.

In a particular preferred embodiment, the frame of the agent delivery device may include a part which is configured or designed to remain largely outside of or external to the nostrils in use. This part of the frame thus preferably incorporates the handle portion described above. This ‘external’ part of the frame may interconnect the frame members on which the respective delivery elements are provided or supported. To this end, this ‘external’ part of the frame may include a generally U-shaped body comprising a pair of leg members, each of which connects to the respective frame members with the delivery elements. In use, therefore, the U-shaped body is arranged to span a septum of a nose when worn by an individual, with the leg members extending into each nostril on either side of the septum. The distal end regions of the leg members may be arranged, in use, to engage with the septum and extend from the septum behind the columella and alar fibrofatty tissue of the nose, allowing the frame members on which the delivery elements are carried, in use, to extend along respective nasal orifices to an inner wall of the nostrils.

In a preferred embodiment, the frame includes a sensory comforter to promote comfort of the individual during use of the device. To this end, the sensory comforter may comprise a source of a substance for inhalation by the individual to promote the comfort or user experience of the individual. The source is preferably supported on the frame, e.g. as a pad impregnated with the substance to be inhaled, and is configured to be positioned adjacent the individual's nostrils when each frame member on which a respective delivery element is provided or carried is received and accommodated within a nostril. Thus, the sensory comforter is preferably provided on the external part of the fame, e.g., the handle portion or the U-shaped body of the frame.

In a preferred embodiment, the frame, or each said at least one frame member, forms a substrate of the device and is comprised of a polymer plastic material, which is preferably selected from the group consisting of polypropylene (PP), polyethylene (PE), polystyrene (PS), polyamide (PA) e.g., Nylon, and/or styrene-ethylene-butylene-styrene (SEBS). Alternatively, or in addition, the substrate of the device (i.e., the frame and/or each said at least one frame member) may be comprised of metal wire.

In a preferred embodiment, the substrate or frame of the agent delivery device comprises a framework structure, such as an open framework structure, preferably in the form of or comprising an array, web, or mesh of strand-like elements or filaments. The elements, strands, or filaments of the framework structure or of the array, web, or mesh preferably comprise or are formed of a polymer plastic material and may be fused or bonded together to form the substrate/frame of the agent delivery device. In this regard, the framework structure is preferably formed in an additive manufacturing or ‘3D printing’ process. The framework structure is desirably relatively soft and flexible in the hand of a user.

In a preferred embodiment, the delivery means or the delivery element provided on or carried by the substrate/frame of the agent delivery device for delivering the agent to the nasal tissue may comprise an open framework, preferably formed as or comprising an array, web, or mesh of strand-like elements or filaments, which may comprise an outer layer or outer covering on the substrate or frame of the device. The elements, strands, and/or filaments of the open framework, array, web, or mesh comprising the delivery means or delivery element are typically formed of a polymer plastic material and may be fused or bonded together. In this regard, the open framework comprising the delivery means or delivery element may be formed in an additive manufacturing or 3D printing process. The open framework or the array, web or mesh of strands or filaments in the delivery means or delivery element is desirably very soft and flexible in the hand of an individual user and very soft to the touch upon insertion into the nasal cavity. This promotes user comfort and avoids the risk of tissue damage during the use. The open framework structure of each delivery means or delivery element creates pores or apertures for delivering the agent therethrough to the tissue or mucosa of the nasal cavity. Naturally, they may cover a space and voids below filed with filaments and/or fibres that form a supply or reservoir of the agent to be delivered, especially for a gel or viscous liquid. The filaments or fibres may promote transfer of the agent from the supply to the nasal tissue of the wearer by ‘wicking’ or capillary action. In this way, the open filament framework can promote enhanced transfer of the agent to the nasal tissue by the delivery element during the predetermined time period for agent delivery.

In a preferred embodiment, the agent delivery device is provided with a unique identifier for use in recording or registering an individual to whom the agent is delivered with the device. In this way, the unique identifier is designed to support data integrity and tracking of device usage. The unique identifier may be in the form of a number, a symbol, a code, a signal, or any other form suitable for creating a unique identity for the device. The unique identifier may be physically provided in or on the device itself, e.g., in or on the substrate or frame of the device, or in association with the device; e.g., in or on packaging.

In a preferred embodiment, the unique identifier is adapted to be recognised or recorded automatically when registering the individual to whom an agent is delivered with the device. In one particular preferred embodiment, the unique identifier is provided in the form of a code (e.g., a QR code) which is able to be scanned or read in an automated way e.g., via QR code-recognition software in a mobile phone application. That code may be provided on the device itself or on the packaging associated with the device. In another particular preferred embodiment, the unique identifier is provided in the form of a signal, e.g., a radio-frequency signal via an RFID device, which is able to be scanned or read in an automated manner. A signal emitter (e.g., RFID) for emitting the unique identifier signal may again optionally be provided in or on the device itself. Once the unique identifier has been scanned or read to record the specific agent delivery device, personal details of the individual to whom the agent is delivered with that device may then be registered (e.g., name, address, gender, date of birth, health insurance details, etc.) to a database.

According to a further aspect, the present disclosure provides an agent delivery kit, comprising an agent delivery device according to any of the embodiments described above for delivering an agent with the nose; and an agent container for re-supplying the or each delivery element, desirably separated or removed from the frame of the agent delivery device.

In a preferred embodiment, the agent container holds a supply of the agent (e.g., a liquid or gel agent) for re-supplying the or each delivery element by immersing the or each delivery element therein.

According to another aspect, the disclosure provides an agent delivery system, comprising an agent delivery device or kit according to any of the embodiments described above for delivering an agent; and a software application for supporting use of the agent delivery device by an individual.

In a preferred embodiment, the software application is accessible or operable via a mobile telecommunications device (herein simply “mobile device”), such as a mobile phone or tablet. In this regard, the software application could optionally be downloaded and installed on the mobile device or alternatively could be accessible online via a web browser. The software application comprises a computer program or computer software configured to be executed by a computer processor, e.g., microprocessor, as is typically found within a mobile computing device, such as a mobile phone or a tablet. Of course, the computer program or computer software will also be able to be executed by a personal computer, such as a laptop computer, of the individual. The software application may be available to a user online (e.g. via a cloud server) as a computer program product to be downloaded or alternatively accessible via an Internet browser.

In a preferred embodiment, the software application provides instructions to the individual via a mobile device on correct use of the agent delivery device. The instructions may be available in a number of languages to be selected by the individual and/or may be provided in a schematically illustrative manner which may be understood irrespective of language. The software application may include a timer to provide a means for indicating to the individual when a predetermined agent delivery time has elapsed. The timer will typically be started after the delivery elements of the agent delivery device have been introduced into the nostrils of the individual in accordance with the instructions provided. The time may include an alarm which sounds via the mobile device to alert the individual at the end of the predetermined time period for agent delivery. This can also operate to record that the individual has complied with the agent delivery process.

According to yet another aspect, the disclosure provides a method of delivering an agent to an individual, the method comprising steps of:
providing an agent delivery device comprising a frame, at least part of which is configured to be received and retained within a nostril of the individual, and a delivery element provided on and/or carried by said part of the frame for delivering the agent;
introducing the delivery element provided on and/or carried by said part of the frame into a nostril of the individual, whereby it is received and retained within the nostril such that the delivery element contacts or engages with an inner surface or tissue of the nostril to deliver the agent; and
allowing the delivery element provided on and/or carried by said part of the frame to remain or reside within the nostril for a predetermined period of time for delivery of the agent.

In a preferred embodiment, the predetermined period of time for agent delivery is at least about 2 to 3 seconds, preferably at least about 10 to 30 seconds, more preferably at least one or two minutes, and optionally in the range of about 5 minutes to about 8 hours, e.g. in the range of about 15 minutes to about 4 hours. In this regard, the individual typically wears the agent delivery device for the predetermined period of time. Thus, the delivery element is typically introduced into and resides within an anterior nasal cavity, e.g. in the nares or nasal vestibule, to deliver the agent nasally for the predetermined period of time.

In a preferred embodiment, the method includes providing or conveying the agent from a supply, such as a reservoir, on the frame of the device to the or each delivery element during the predetermined period of time. In this way, the method may comprise the step of replenishing or recharging the/each delivery element during use of the device.

In a preferred embodiment, the method comprises accessing and/or operating a software application-preferably via a mobile device, such as a mobile phone or tablet—to support use of the agent delivery device by an individual. In this regard, the software application may be downloaded and installed on the mobile device or alternatively may be accessed online via a web browser.

According to a further aspect, the present disclosure provides a frame of an agent delivery device for delivering a therapeutic or medicinal agent to an individual. The frame of the agent delivery device includes at least one frame member adapted or configured to be received and retained or accommodated within a nostril of the individual. The at least one frame member is adapted or configured to carry or to support delivery means, such as a delivery element, for delivering the agent such that, when the frame member is retained or accommodated within a nostril, the delivery means/delivery element contacts or engages an inner wall or surface (e.g., the tissue) of the nostril to deliver the agent to the individual, preferably percutaneously.

### BRIEF DESCRIPTION OF THE DRAWINGS

For a more complete understanding of the disclosure and advantages thereof, exemplary embodiments of the disclosure are explained in more detail in the following description with reference to the accompanying drawing figures, in which like reference signs designate like parts and in which:

FIG. 1 is a schematic perspective view of an agent delivery device according to a first preferred embodiment;

FIG. 2 is a perspective view of an agent delivery device according to the first preferred embodiment;

FIG. 3 is a schematic perspective view of an agent delivery device according to a second preferred embodiment;

FIG. 4 is a perspective view of an agent delivery device according to a second preferred embodiment;

FIG. 5 is a perspective view of an agent delivery device according to a third preferred embodiment;

FIG. 6 is a perspective view of a frame of an agent delivery device according to a fourth preferred embodiment;

FIG. 7 is a perspective view of an agent delivery device according to a fifth preferred embodiment;

FIG. 8 is a perspective view of an agent delivery device according to a sixth preferred embodiment;

FIG. 9 is a schematic perspective view of an agent delivery device pursuant to a seventh preferred embodiment;

FIG. 10 is another schematic perspective view of the agent delivery device in FIG. 9;

FIG. 11 is a perspective view of a frame of an agent delivery device according to an eighth preferred embodiment;

FIG. 12 is a side view of the agent delivery device of the embodiment in FIG. 10;

FIG. 13 is a schematic illustration of the agent delivery device of FIG. 11 in use;

FIG. 14 is a schematic perspective view of an agent delivery device according to a ninth preferred embodiment;

FIG. 15 is a schematic perspective view of an agent delivery device according to a tenth preferred embodiment;

FIG. 16 is a schematic perspective view of an agent delivery device according to an eleventh preferred embodiment;

FIG. 17 is another schematic perspective view of the agent delivery device in FIG. 16;

FIG. 18 is a schematic perspective view of an agent delivery device according to a twelfth preferred embodiment; and

FIG. 19a and FIG. 19b are schematic perspective views of two delivery elements of the agent delivery device of the embodiment in FIG. 18.

The accompanying drawings are included to provide a further understanding of the present disclosure and are incorporated in and constitute a part of this specification. The drawings illustrate particular embodiments of the disclosure and together with the description serve to explain the principles of the present disclosure. Other embodiments and many of the attendant advantages will be readily appreciated as they become better understood with reference to the following detailed description.

It will be appreciated that common and/or well understood elements that may be useful or necessary in a commercially feasible embodiment are not necessarily depicted in order to facilitate a more abstracted view of the embodiments. The elements of the drawings are not necessarily illustrated to scale relative to each other. It will also be understood that certain actions and/or steps in an embodiment of a method may be described or depicted in a particular order of occurrences while those skilled in the art will understand that such specificity with respect to sequence is not actually required.

### DETAILED DESCRIPTION OF THE EMBODIMENTS

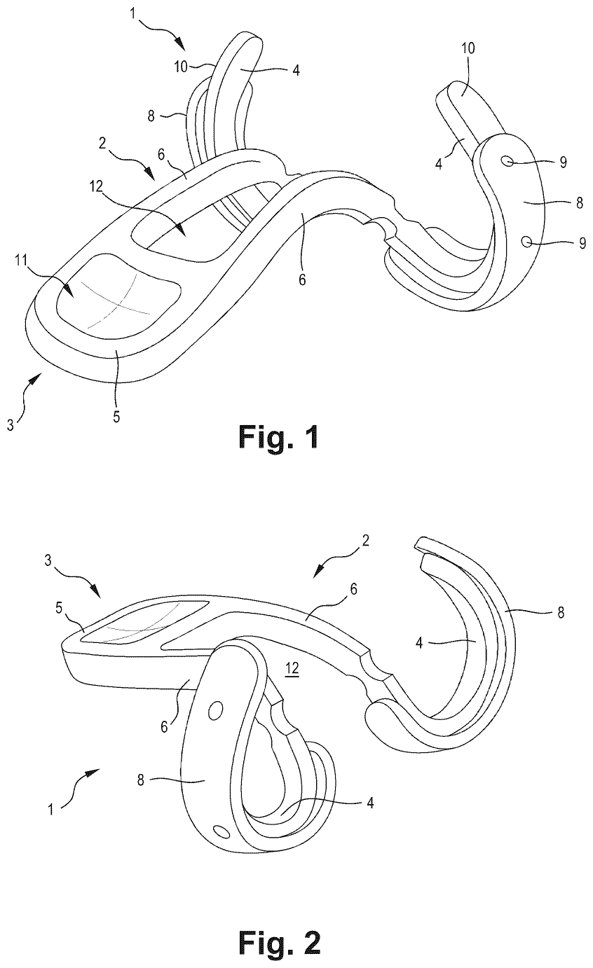

Referring initially to FIGS. 1 and 2 of the drawings, a first preferred embodiment of an agent delivery device 1 for delivering an agent, especially a medicinal or therapeutic agent, to an individual nasally according to the disclosure is shown in perspective views. The agent delivery device 1 comprises a frame 2 formed of a polymer plastic material, such as, for example, polypropylene (PP), polyethylene (PE), polyamide (PA) e.g., Nylon, polystyrene (PS), styrene-ethylene-butylene-styrene (SEBS), or like. The frame 2 may be formed by moulding and, in this case, is formed as an integral or unitary component. As is apparent from FIG. 1 and FIG. 2, the frame 2 of the device 1 comprises a central portion 3 and two cantilevered frame members 4 in the form of ribs which are curved (like “cow horns”) and extend laterally outwardly in opposite directions from the central portion 3. The central portion 3 is part of the frame 2 which, in use, is configured or designed to remain largely outside of or external to the nostrils. The central portion 3 comprises a generally U-shaped body having a tab part 5 and two frame members 6 in the form of stems or legs extending roughly parallel to one another from the tab part 5 towards and into connection with a respective one of the lateral curved rib members 4.

With reference to FIGS. 1 and 2 of the drawings, the agent delivery device 1 further comprises a delivery element 8 in the form of a pad of fibrous delivery material provided on and carried by each of the curved rib members 4. Each delivery pad 8 is comprised, for example, of cotton, rayon, calcium alginate, polyester, polypropylene, or polyethylene, and is designed for holding and delivering a liquid or gel agent, typically percutaneously via contact with the nasal tissue or mucosa. Each delivery pad 8 is mounted or connected to a respective one of the rib members 4 at connection points 9 by bonding, fusing, or a connection element, such as a pin connector. Furthermore, each pad 8 extends along a surface or side 10 of the rib member 4 that, in use, is configured to face the tissue of the nasal cavity.

In this regard, it will be appreciated that the pair of rib members 4 with their respective delivery pads 8 are configured to be inserted or introduced into the nostrils of an individual for delivering the agent. To this end, the rib members 4 are designed to be relatively soft and resiliently flexible or ‘springy’ to assist their easy insertion into the nostrils. Similarly, the delivery pads 8 are relatively soft to promote user comfort. The tab part 5 of the central portion 3 forms a handle member for the individual to grasp and to manipulate the device 1 during insertion of the rib members 4 and delivery pads 8 into the nose. As will be understood, it is desirable for the user to avoid touching the delivery pads 8 with his/her hands to avoid any potential contamination of the agent. The individual therefore grasps the device 1 via the tab part 5 of the central portion 3, which may include a curved depression 11 to promote gripping with a finger or thumb. The stem or leg members 6 extending from the tab part 5, and a gap 12 between leg members 6, enable the respective ribs 4 and delivery pads 8 to be inserted into the nostrils on either side of the septum. In this way, the rib members 4 and the delivery pads 8 can be positioned and retained or accommodated within a lower nasal cavity for delivery of the agent via the pads 8. The delivery pads 8 provided on the flexible rib members 4 conform to and complement a surface profile or curvature of the nasal cavity.

In use, therefore, the U-shaped body of the central portion 3 of the agent delivery device 1 is arranged to span a septum of a nose when worn by an individual, with the leg members 6 extending into each nostril on either side of the septum. The leg members 6 may be inclined somewhat towards each other such that a relatively greater distance is provided between the leg members 6 at the tab part 5 to accommodate the columella of the nose when worn by the individual. The distal end regions of the leg members 6 may, in use, be arranged to engage with the septum and extend from the septum behind the columella and alar fibrofatty tissue of the nose, thereby allowing the rib members 4 on which the delivery elements 8 are carried, in use, to extend along respective nasal orifices to an inner wall of the nostrils.

After the rib members 4 and the delivery pads 8 of agent delivery device 1 are introduced and accommodated in the nostrils of the individual, they remain or reside there for an extended or predetermined agent delivery time of least a few seconds, preferably at least about 30 seconds, and more typically from one or two minutes to about 15 minutes and up to several hours. For example, the individual may wear the agent delivery device 1 during the day or night (e.g., while he/she sleeps) during delivery of the agent via the pads 8. After the predetermined period of time for agent delivery has elapsed, the user may remove the rib members 4 and delivery pads 8 from the nose by grasping the handle or tab part 5 and gently withdrawing those parts of the device 1 from the nostrils.

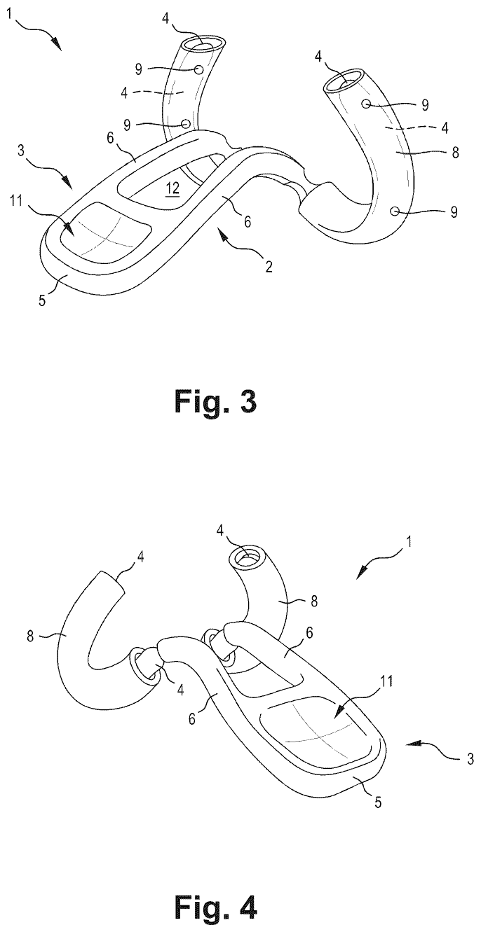

With reference now to drawing FIGS. 3 and 4, a second preferred embodiment of an agent delivery device 1 for delivering the agent nasally to an individual according to the disclosure is shown in perspective views. The general structure of the agent delivery device 1 in this embodiment is essentially the same as in the first embodiment, so another description of that structure will not be repeated, but like reference characters in the drawings identify like parts. A difference in this second embodiment, however, concerns the delivery elements 8 which are provided in the form of tubular sleeves, which surround or sheathe the curved rib members 4, instead of the pads shown in FIGS. 1 and 2. In this embodiment, the delivery sleeves 8 may be knitted sleeves comprised of fibres of e.g. cotton, rayon, calcium alginate, polyester, polypropylene, or polyethylene, or may also be foam sleeve elements comprised of e.g. a urethane foam. The delivery sleeves 8 may be form-fitted or friction fitted on the flexible rib members 4 or they may optionally again be connected to the rib members 4 via connection points 9, e.g. by bonding, fusing, or a connection element, like a pin connector. Use and operation of the agent delivery device 1 of the second embodiment correspond to the use and operation described above for the first embodiment.

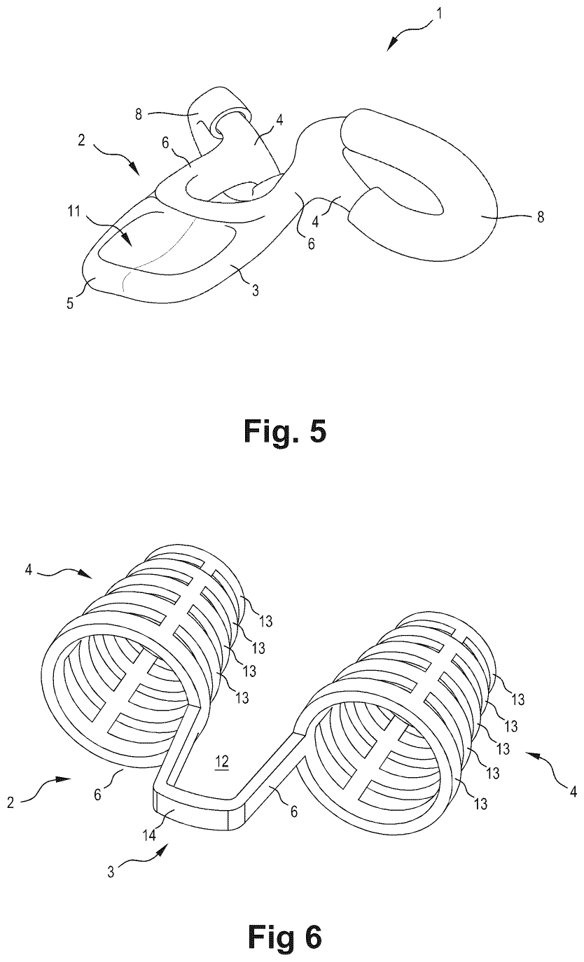

FIGS. 5, 6 and 7 of the drawings illustrate a number of alternative embodiments of an agent delivery device 1 that exhibit differences in the configuration of the frame 2. Again, however, it will be noted that the general use of these agent delivery devices 1 corresponds to the use as it has been described above for the first embodiment. In the third embodiment of FIG. 5, the frame members 4 upon which the delivery elements 8 are provided or carried are in the form of closed loops. Like for the second embodiment of FIGS. 3 and 4, the delivery elements 8 are provided in the form of sleeves which surround or sheathe the loop members 4. In a fourth embodiment of FIG. 6, the frame members 4 upon which respective delivery elements (not shown) are to be supported are provided in the form of a series of interconnected loops or hoops 13, which form a cage structure. The delivery elements in this case could be provided in the form of a cylindrical sheath or sleeve for covering an outer periphery of the cage structure. This structure results in the delivery elements (not shown), in use, extending further or deeper into the nasal cavity. In this embodiment, also, the tab part 5 is missing and the central portion 3 comprises a simple band 14 interconnecting the leg members 6.

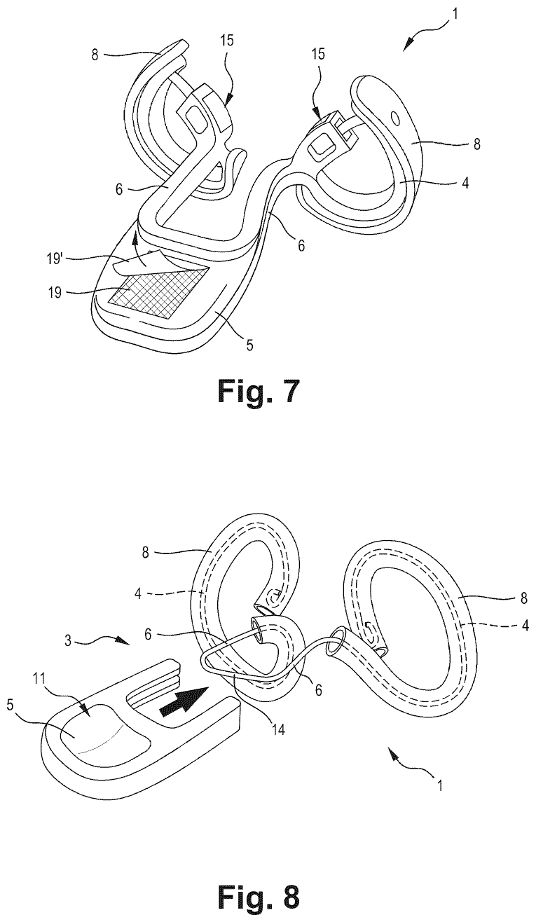

In the fifth embodiment shown in FIG. 7, the agent delivery device 1 includes a frame 2 having a very similar configuration to that of the first embodiment. The delivery elements 8 are also provided in the form of pads, as in the first embodiment. In this fifth embodiment, however, each rib member 4 includes an adjuster 15 for setting or adjusting a radial position of the delivery pad 8. In this regard, the adjuster 15 preferably comprises a mechanism, e.g. a ratchet mechanism, for setting or adjusting a curvature or radial extent of the respective rib member 4 and, thus, also of the associated delivery pad 8. In this embodiment, for example, each adjuster mechanism 15 comprises a pin 16 and a socket 17 arranged to receive and engage the pin 16. An enlargement or head may be provided at an end of the pin 16 to prevent the pin withdrawing from the socket 17. In this regard, a shoulder provided inside the socket 17 may be configured to engage with the enlargement or head of the pin 16 to prevent the pin from withdrawing from the socket.

This may allow some pre-adjustment of the shape and/or position of the rib members 4 and the delivery pads 8 prior to insertion of those parts of the device 1 into the individual's nostrils for agent delivery. Furthermore, this embodiment includes a timing indicator 19 for indicating to the individual or wearer when a predetermined time period for the agent delivery has elapsed. The indicator 19 is provided on the tab part 5 of the frame 2 that remains external of the nostrils, and therefore visible, during use of the device 1. The timing indicator 19 comprises a patch that is configured to change appearance (colour) over time. In this regard, the indicator patch 19 includes a substance selected to react with air and/or light over the predetermined time period in order to change colour over that time. Upon commencing use of the device 1, therefore, the indicator patch 19 is initially exposed to air and/or light by removing a protective cover or label 19′. After the predetermined time for agent delivery has elapsed, the indicator patch 19 will have changed colour due to exposure to air and/or light and will indicate to the user or wearer that the agent delivery device 1 can now be removed. In this way, the device 1 is designed to achieve consistency of use by individuals testing themselves. This feature of the timing indicator 19 may be included in any of the other embodiments.

With reference now to FIG. 8 of the drawings, a sixth embodiment of an agent delivery device 1 is illustrated. This sixth embodiment corresponds substantially to the embodiment of FIG. 7, except that, in place of the timing indicator 19, it includes a sensory comforter 18 to promote sensory comfort during agent delivery. The sensory comforter 18 comprises a pad impregnated with a substance for inhalation by the individual. The substance preferably provides a pleasant aroma to mask or offset the agent provided on the delivery pads 8 thereby to promote user comfort. In this way, the pad 18 provides a source of the sensory comforter supported on the central portion 3 of the frame 2, which is positioned adjacent the individual's nostrils when the rib members 4 and respective delivery pads 8 are accommodated or retained within the nostrils in use. The sensory comforter 18 emits the substance in the form of fumes, vapour, or volatiles V which, upon inhalation, promote a pleasant sensory experience for the user and thus operate to offset or mask any unpleasant sensory experience from the agent itself during the delivery time period. This feature of the sensory comforter 18 may be included in any of the other embodiments.

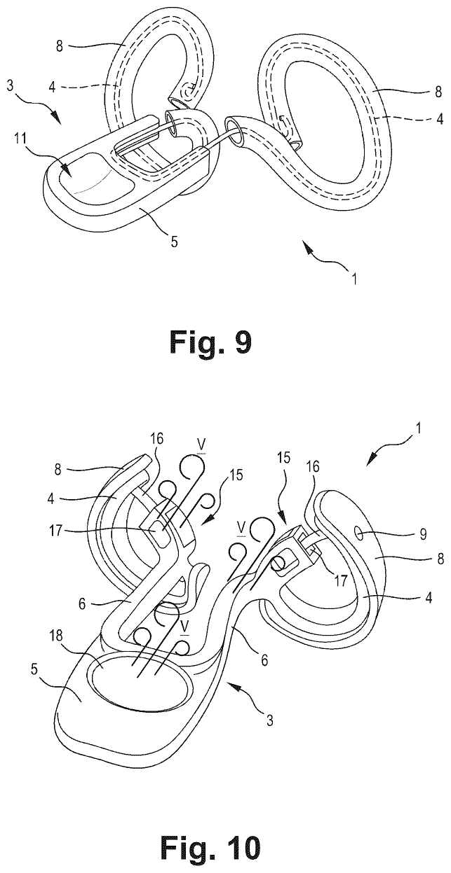

With reference to further FIGS. 9 and 10 of the drawings, a seventh preferred embodiment of an agent delivery device 1 is shown schematically. In this embodiment, the frame 2 of the device 1 is comprised of a metal wire. The wire is shaped to provide a U-shaped central portion 3 and a pair of frame members 4 in the form of ribs which are curved and extend laterally outwardly in opposite directions from the central portion 3. Again, the rib members 4 are resiliently flexible and ‘springy’ for ready adaptation to the size and shape of the nasal cavity of the individual, thereby promoting wearer comfort. In this example, the delivery elements 8 are again provided in the form sleeves which surround or sheathe the rib members 4. The central portion 3 of this embodiment includes a removable tab part or handle 5, preferably formed of plastic, which is designed to clip onto two roughly parallel extending wire leg members 6 and a connecting wire band 14 of the central portion 3 interconnecting the two rib members 4. The fact that the tab part or handle 5 is removable makes the agent delivery device 1 considerably less obtrusive when it is being worn by the user, e.g., when sleeping.

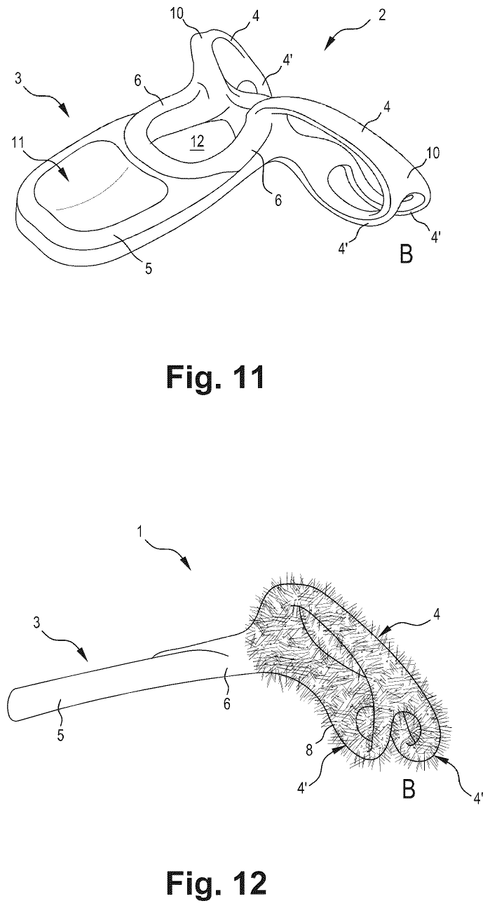

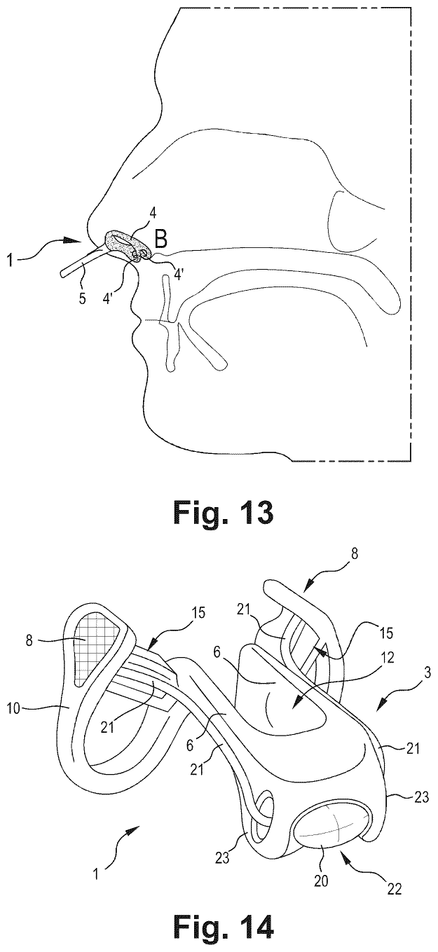

Referring now to drawing FIGS. 11 to 13, an eighth preferred embodiment of an agent delivery device 1 is illustrated. As can be seen in FIG. 11, the central portion 3 of the frame 2 is very similar in configuration to the previous embodiments and includes a generally U-shaped body having a tab part 5 forming a handle and a pair of leg members 6 extending therefrom. The rib members 4 in this embodiment are somewhat different, however. Each elongate rib member 4 has looped profile which approximates an ellipse and enables the rib member 4 to be received and to seat within the nostril comfortably and consistently in the nasal vestibule, as seen in FIG. 13, which is important for achieving consistency both in use and in agent delivery. Further, each rib member 4 on which the delivery means 8 is provided/carried in form of a layer of fibres, especially flocked fibres, is configured broader or wider in a region B thereof to be located deeper within the nasal cavity or nares. In this way, the rib member 4 provides a larger surface area for bringing the delivery means 8 carried thereon into contact with the surface of the nares or nasal cavity likely to provide greater agent load delivery. For similar reasons, the rib member 4 is open, splayed or divided into riblets 4′ in this region to provide greater surface area for supporting the fibres, e.g. flocked fibres, of the delivery means 8, to increase a potential agent load.

With reference now to FIG. 14 of the drawings, a ninth embodiment of an agent delivery device 1 is shown in a schematic perspective view. In this example, the frame 2, and particularly the rib members 4 having an adjuster 15 for setting or adjusting a radial position of each delivery pad 8 of the device 1, is quite similar to the embodiments shown in FIG. 7 and FIG. 10. In will be noted, however, that the delivery pads 8 in this case do not extend over the whole surface or side 10 of the rib member 4, but rather are confined to an enlarged end region 7 of each rib member 4. The delivery pads 8 themselves are comprised of a porous and relatively soft, spongy foam material. Another difference in this embodiment resides in the configuration of the central portion 3 of the frame 2. While the central portion 3 again comprises a generally U-shaped body with a gap 12 between leg members 6 to enable the respective ribs 4 and delivery pads 8 to be inserted into the nostrils of a wearer on either side of the septum, the front end of the central portion in this embodiment is not formed as a flat tab. Instead, the front end of the central portion 3 incorporates a receptacle 20 which forms a supply or reservoir of liquid agent. Tubing or fluid lines 21 provide fluid communication between the receptacle or reservoir 20 and the spongy deliver pads 8. Further, the receptacle 20 is formed from a flexible and deformable material (e.g., a polymer plastic material) and is mounted in the central portion 3 of the frame 2 within a recess 22 that enables access to the receptacle 20 by a thumb or finger of the wearer or user of the device 1. When the device 1 of FIG. 14 is used for the first time, the liquid agent may be sealed inside the capsule-like receptacle 20. The agent may, for example, be released to flow from the receptacle 20 towards delivery pads 8 by inserting ends of the fluid lines 21 through side apertures 23 of central portion 3 to pierce the capsule-like receptacle 20. The fine tubing (e.g., microtubes) of fluid lines 21 may in any case promote conveyance of the liquid agent from the receptacle 20 via capillary action. By applying manual pressure to the flexible receptacle 20 (e.g. by thumb or finger) the liquid agent can also be forced along the fluid lines 21 to the delivery pads 8. In an alternative embodiment, the tubing or fluid lines 21 may be fixed in fluid communication with the capsule-like receptacle 20 and applying pressure to or squeezing the reservoir via the recess 22 may break an internal seal (not shown) and cause the liquid agent to flow to the delivery pads 8.

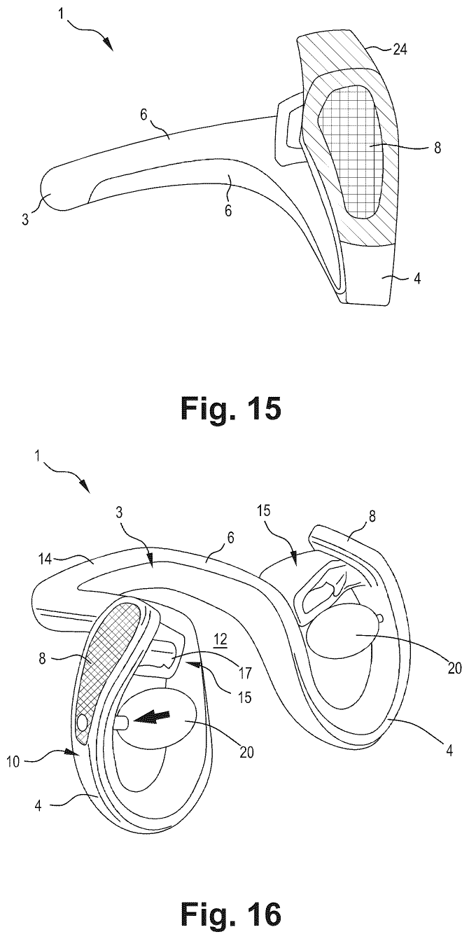

Referring to drawing FIG. 15, a tenth embodiment of an agent delivery device 1 is illustrated, which may incorporate any of the features of the preceding embodiments. In this case, the delivery elements 8 are again provided as pads at a distal end region 7 of the rib members 4, but the delivery pads 8 have a recessed region for receiving and/or absorbing the agent. This embodiment also includes a protective cover or film layer 24 which is to be removed (e.g., peeled away) by the individual before using the device 1.

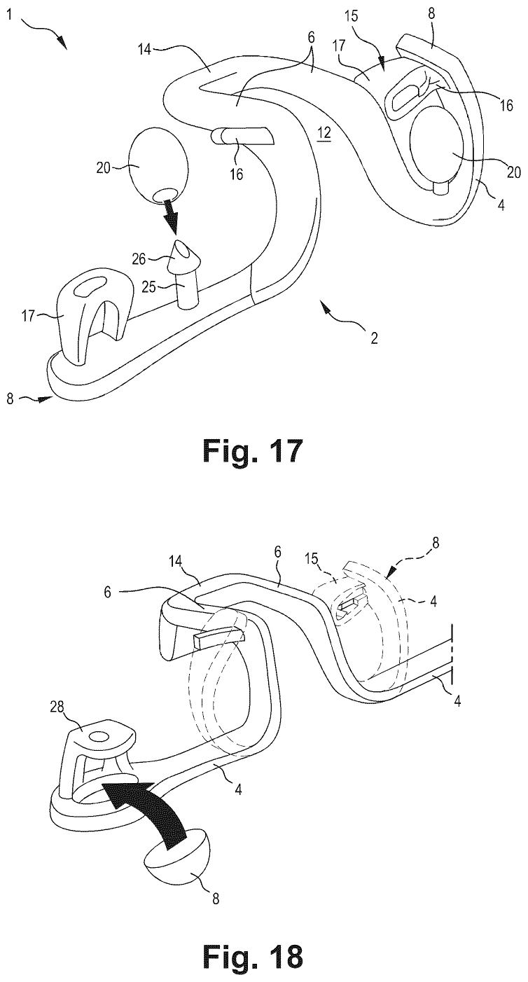

With reference to drawing FIGS. 16 and 17, an eleventh embodiment of an agent delivery device 1 is shown in schematic perspective views. The agent delivery device 1 in this case is similar to the embodiment in FIG. 14. The delivery pads 8 are again provided at the end regions 7 of the curved rib members 4 but they extend somewhat further along the outer side 10 of those rib members 4. Furthermore, while the agent delivery device 1 of this embodiment again includes a supply of the liquid agent in communication with the delivery pads 8, that supply is not incorporated in the central portion 3 of the frame 2. Rather, the central portion of the frame 2 simply comprises a U-shaped body with legs 6 interconnected by a band 14. Instead, the agent delivery device 1 has a separate capsule-like receptacle 20 for connection to each rib member 4 in fluid communication with the respective delivery pad 8 on that rib member 4. In this regard, each rib member 4 includes a hollow pin or spike 25, preferably having a pointed and optionally barbed end 26, for piercing and receiving a respective one of the capsule-like receptacles 20, as shown by the arrow in FIG. 17. In this regard, each capsule-like receptacle 20 comprises a relatively thin plastic shell filled with the liquid agent to be delivered to the individual. By mounting the capsule 20 on the hollow pin or spike 25, the liquid agent may flow through the hollow pin or spike into the adjacent pad 8 with which it is connected in fluid communication. This flow of the agent to soak or impregnate each delivery pad 8 may be assisted by the user gently squeezing the respective capsule-like receptacle after it is mounted on the pin or spike 25. The capsules 20 may be disposable and may be replaceable on the device 1 after the contents of a first pair of capsules 20 have been fully delivered to the user.

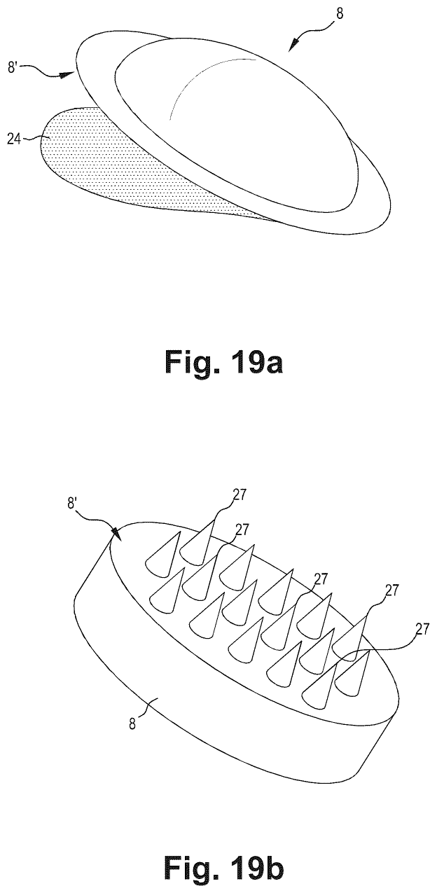

Further, with reference to drawing FIGS. 18, 19a and 19b, a twelfth embodiment of an agent delivery device 1 is shown. In this embodiment, the delivery elements 8 of the device 1 provided at the end regions 7 of the rib members 4 are in the form of replaceable cartridge elements. In particular, these cartridge delivery elements 8 contain a supply of the agent to be delivered, e.g., within a built-in receptacle or chamber, and provide and interface 8′ for contact with an inner surface of the nostrils at which interface 8′ the transfer or delivery of the agent to the user takes place. The interface 8′ may once again comprise a pad or layer of a porous foam or fibrous material which is impregned with the agent for delivering the agent to the wearer. Alternatively, or in addition, the interface 8′ may include an array of micro-needles 27 for superficial penetration of the skin for enhanced delivery of the agent by percutaneous absorption. The micro-needles may be designed to dissolve slowly upon contact with the skin. The interface 8′ is preferably covered by a protective cover or film layer 24 which is to be removed before use. As can be seen in FIG. 18, the end region 7 of each of the rib members 4 is provided with a holder 28, such as a clip, for releasably mounting a respective cartridge delivery element 8. As with the capsules 20 in the embodiment of FIGS. 16 and 17, the cartridges 8 may be disposable and replaceable on the device 1 after the contents of a first pair of cartridges 8 have been fully delivered to the user/wearer.

Although specific embodiments of this disclosure are illustrated and described herein, it will be appreciated by persons of ordinary skill in the art that a variety of alternative and/or equivalent implementations exist. It should be appreciated that each exemplary embodiment is an example only and is not intended to limit the scope, applicability, or configuration in any way. Rather, the foregoing summary and detailed description will provide those skilled in the art with a convenient road map for implementing at least one exemplary embodiment, it being understood that various changes may be made in the function and arrangement of elements described in an exemplary embodiment without departing from the scope as set forth in the appended claims and their legal equivalents. Generally, this application is intended to cover any adaptations or variations of the specific embodiments discussed herein.

It will also be appreciated that the terms “comprise”, “comprising”, “include”, “including”, “contain”, “containing”, “have”, “having”, and any variations thereof, used in this document are intended to be understood in an inclusive (i.e. non-exclusive) sense, such that the process, method, device, apparatus, or system described herein is not limited to those features, integers, parts, elements, or steps recited but may include other features, integers, parts, elements, or steps not expressly listed and/or inherent to such process, method, process, method, device, apparatus, or system. Furthermore, the terms “a” and “an” used herein are intended to be understood as meaning one or more unless explicitly stated otherwise. Moreover, the terms “first”, “second”, “third”, etc. are used merely as labels, and are not intended to impose numerical requirements on or to establish a certain ranking of importance of their objects. In addition, reference to positional terms, such as “lower” and “upper”, used in the above description are to be taken in context of the embodiments depicted in the figures, and are not to be taken as limiting the disclosure to the literal interpretation of the term but rather as would be understood by the skilled addressee in the appropriate context.

Any discussion of background art in the present disclosure should in no way be considered an admission that such background is prior art, nor that such background is widely known or forms part of the common general knowledge in the field in Australia or in any other country worldwide.

1. An agent delivery device for delivering an agent, preferably a medicinal agent or a therapeutic agent, to an individual, the delivery device comprising: a substrate configured to be received and retained or accommodated within a nostril of the individual; and a delivery element provided on and/or carried by the substrate for receiving and delivering the agent; wherein the delivery element is provided on and/or carried by the substrate such that, when the substrate is retained or accommodated within a nostril, the delivery element contacts or engages with tissue of the nostril to deliver the agent to the individual.

2. An agent delivery device according to claim 1, wherein the substrate forms a frame of the device for supporting the delivery element provided and/or carried thereon; wherein the frame preferably comprises at least one frame member configured to be received and retained or accommodated within the nostril of the individual, and wherein the delivery element is provided on and/or carried by the frame member.

3. An agent delivery device for delivering an agent, preferably a medicinal agent, to an individual nasally, the agent delivery device comprising:
a frame having at least one frame member configured to be received and retained or accommodated within a nostril of the individual; and
a delivery element provided on and/or carried by the at least one frame member for receiving and delivering the agent, preferably in the form of a liquid or gel;
wherein the delivery element is provided on and/or carried by the frame member such that, when the frame member is retained or accommodated within a nostril, the delivery element contacts and/or engages with an inner surface or tissue of the nasal cavity to deliver the agent to the individual.

4. An agent delivery device according to any one of claims 1 to 3, wherein the device is wearable or configured to be worn by the individual such that the delivery element is received and accommodated within the nostril of the individual for an extended or predetermined agent delivery time.

5. An agent delivery device according to claim 4, wherein the agent delivery time is at least 2 seconds, preferably at least about 30 seconds, optionally in the range of about 2 minutes to about 8 hours, e.g., in the range of about 10 minutes to about 4 hours.

6. An agent delivery device according to any one of claims 2 to 5, wherein the at least one frame member on which the delivery element is provided or carried is configured to be received and retained or accommodated within a lower nasal cavity, e.g. within a nasal vestibule, for delivering the agent, wherein the at least one frame member is formed to complement a surface profile or curvature of the nasal cavity, and wherein the delivery element is provided or carried on a surface or region of the frame member that faces the tissue of the nasal cavity.

7. An agent delivery device according to claim 6, wherein the frame member is both elongate and resiliently flexible and is biased into contact with the nasal cavity so that the delivery element bears against and/or engages with tissue of the nasal cavity when the frame member is accommodated within a nostril.

8. An agent delivery device according to any one of claims 2 to 7, wherein the frame comprises a pair of frame members, each of the frame members configured to be accommodated within a respective one of the nostrils of the individual, wherein the device comprises a respective delivery element provided on or carried by each of the frame members such that each delivery element is configured, in use, to bear against or engage with tissue of the respective nostril to deliver the agent.

9. An agent delivery device according to any one of claims 2 to 8, wherein the or each frame member has a curved or looped configuration.

10. An agent delivery device according to any one of claims 1 to 9, wherein the delivery element comprises a delivery material for receiving and delivering the agent, the delivery material being comprised of fibres and/or a polymer foam, with the fibres preferably selected from the group of flocked fibres, compressed fibres, fibre sheet, and knitted fibres, wherein the agent is impregnated on the delivery material for delivery to the individual, for example, by percutaneous absorption.

11. An agent delivery device according to claim 10, wherein the delivery material is comprised of fibres and is selected from the group consisting of cotton, rayon, calcium alginate, polyester, polypropylene, polyamide (Nylon) and polyethylene, and/or wherein the delivery material is comprised of polymer foam and comprises urethane.

12. An agent delivery device according to any one of claims 1 to 11, wherein the or each delivery element is configured in the form of a pad, preferably an elongate pad, provided or carried on the respective frame member.

13. An agent delivery device according to any one of claims 1 to 12, wherein the or each delivery element comprises a plurality of micro-needles projecting from a surface thereof for contact and/or engagement with an inner surface or tissue of the nasal cavity to promote delivery of the agent; for example, percutaneously.

14. An agent delivery device according to any one of claims 1 to 13, comprising a supply of the agent on the substrate or frame of the device, wherein the supply is in communication with the delivery means or delivery element, preferably in fluid communication, for providing or conveying the agent thereto.

15. An agent delivery device according to claim 14, wherein the supply of the agent includes a reservoir for holding an amount of the agent, preferably in a liquid or gel form, but optionally in a film-like or a powder form, and wherein the reservoir is connected in fluid communication with the delivery means or the delivery element for conveying the agent thereto from the reservoir.

16. An agent delivery device according to claim 15, wherein the reservoir for holding an amount of the agent is removably mounted or replaceable on the frame of the device for (re)charging the supply of the agent; wherein the reservoir is preferably in the form of a receptacle, such as a capsule, filled with the agent.

17. An agent delivery device according to any one of claims 1 to 16, wherein the frame, or the at least one frame member, is comprised of a plastic material, e.g. polypropylene (PP), polyethylene (PE), polystyrene (PS), polyamide (PA), e.g., Nylon), or styrene-ethylene-butylene-styrene (SEBS), and/or a metal wire.

18. An agent delivery device according to any one of claims 1 to 17, wherein the frame includes a handle portion for manual handling of the agent delivery device by the individual, wherein the or each frame member extends from the handle portion of the frame, the handle portion preferably being releasably connected to the or each frame member.

19. An agent delivery device according to any one of claims 1 to 18, wherein the frame includes a sensory comforter to promote comfort of the individual in use, wherein the sensory comforter comprises a source of a substance for inhalation by the individual to promote comfort of the individual in use, wherein the source is supported on the frame and configured to be positioned adjacent the nostrils when the or each delivery element is accommodated within a nostril.

20. An agent delivery device according to any one of claims 1 to 19, wherein the substrate or frame of the device comprises a framework structure comprising an array, web, or mesh of strand-like elements or filaments, wherein the elements, strands, or filaments of the framework structure preferably comprise a polymer plastic material and are preferably fused or bonded together.

21. An agent delivery device according to claim 20, wherein the framework structure is formed in an additive manufacturing or 3D printing process.

22. An agent delivery device according to any one of claims 1 to 21, wherein the delivery means or delivery element provided on or carried by the substrate or frame of the device for receiving and delivering the agent comprises an open framework and/or an array, web, or mesh of strand-like elements or filaments, that preferably comprises an outer layer or covering on the substrate or frame of the device.

23. An agent delivery device according to claim 22, wherein the elements, strands or filaments of the open framework, array, web, or mesh comprising the delivery means or the delivery element are formed of a polymer plastic material, and are preferably formed in an additive manufacturing or 3D printing process.

24. An agent delivery kit, comprising: an agent delivery device according to any one of claims 1 to 23, and a supply of an agent to be placed on the substrate or frame of the device, the supply being configured to be placed in communication with the delivery means or delivery element for providing or conveying the agent thereto.

25. An agent delivery kit according to claim 24, wherein the agent supply includes a reservoir for holding an amount of the agent, preferably in a liquid or gel form, wherein the reservoir is adapted to be connected in fluid communication with the delivery means or the delivery element for conveying the agent thereto from the reservoir.

26. An agent delivery kit according to claim 25, wherein the reservoir for holding an amount of the agent is in the form of a receptacle, such as a capsule, filled with the agent, wherein the receptable is configured to be mounted on the frame of the device.

27. An agent delivery kit according to claim 25, wherein the reservoir for holding an amount of the agent is configured for charging or supplying the agent in the or each delivery element, preferably by immersing the or each delivery element in the agent.

28. An agent delivery system comprising: an agent delivery device for delivering an agent to an individual according to any one of claims 1 to 23 or an agent delivery kit according to any of claims 24 to 27, and a software application for supporting use of the agent delivery device by an individual.

29. A method of delivering an agent to an individual nasally, the method comprising steps of:
providing an agent delivery device comprising a frame, at least part of which is configured to be received and retained within a nostril of the individual, and a delivery means provided on and/or carried by said part of the frame for receiving and delivering the agent;
introducing the delivery means provided on and/or carried by the said part of the frame into a nostril of the individual, whereby it is received and retained within the nostril, such that the delivery means contacts or engages with an inner surface or tissue of the nostril to deliver the agent; and
allowing the delivery means provided on and/or carried by said part of the frame to remain or reside within the nostril for a predetermined period of time for agent delivery.

30. A method according to claim 29, wherein the predetermined period of time for agent delivery is at least about 2 seconds, preferably at least about 30 seconds, optionally in the range of about 5 minutes to about 8 hours, e.g., in the range of about 15 minutes to about 4 hours; and/or
wherein the agent delivery device is to be worn by the individual for the predetermined period of time; and/or
wherein the delivery means is in the form of an element and is introduced into and resides within the nasal cavity, e.g., the nares or nasal vestibule of the individual, to deliver the agent for the predetermined period of time.
